(19) United States
(12) Patent Application Publication
(10) Pub. No.: US 2020/0256879 A1
(43) Pub. Date: Aug. 13, 2020

(54) METHODS AND COMPOSITIONS FOR PREDICTING AND TREATING INTRACRANIAL ANEURYSM

(71) Applicants: INSERM (Institut National de la Santé et de la Recherche Médicale), Paris (FR); Centre National de la Recherche Scientifique (CNRS), Paris (FR); Université de Nantes, Nantes (FR); Centre Hospitalier Universitaire de Nantes, Nantes (FR)

(72) Inventors: Richard REDON, Nantes - Cedex 1 (FR); Gervaise LOIRAND, Nantes - Cedex 1 (FR); Romain BOURCIER, Nantes - Cedex 1 (FR); Hubert DESAL, Nantes - Cedex 1 (FR)

(21) Appl. No.: 16/758,458

(22) PCT Filed: Oct. 23, 2018
(86) PCT No.: PCT/EP2018/078947

§ 371 Date: Apr. 23, 2020

(30) Foreign Application Priority Data
Oct. 24, 2017 (EP) 17306466.8

(51) Int. Cl. G01N 33/68

(52) U.S. Cl. CPC G01N 33/6893; G01N 2800/50; G01N 2800/329

(57) ABSTRACT

The present invention relates to a method for predicting the risk of having or developing Intracranial aneurysms (IA) in a subject, by identifying at least one mutation in an angiogenic protein, such as Angiopoietin-Like 6 (ANGPTL6). In particular, inventors identified one rare nonsense variant (c.1378A>T) in the last exon of the ANGPTL6 gene which encodes a 10 circulating pro-angiogenic factor mainly secreted from the liver shared by the 4 tested affected members of a large pedigree with multiple IA carriers. They GC showed a 50% reduction of ANGPTL6 serum concentration in heterozygous c.1378A>T carriers compared to non-carrier relatives, due to the non-secretion of the truncated protein produced by the c.1378A>T transcripts. They observed a higher rate of individuals with a history of high blood pressure 15 among affected versus healthy carriers of ANGPTL6 variants, suggesting that ANGPTL6 could trigger cerebrovascular lesions when combined with other risk factors such as hypertension.

### FIELD OF THE INVENTION

The invention is in the field of neurology. More particularly, the invention provides methods and compositions to predict and treat intracranial aneurysms (IA).

### BACKGROUND OF THE INVENTION

Intracranial aneurysms (IA) are acquired cerebrovascular abnormalities affecting 3% of the general population [mean age 50 years] (1). They are characterized by a localized dilation and wall thinning in typical locations in intracranial arteries (2). The most notorious and deleterious complication of an IA is the rupture, resulting in subarachnoid haemorrhage that can lead to severe disability and death (3). Unfortunately, there are neither reliable biomarkers nor diagnostic tools to predict the formation and/or the evolution of an IA in any given individual. Current treatments are more or less invasive (microsurgical or endovascular treatment) with a risk of procedural morbidity/mortality (4).

Although the pathogenesis of IA has been the subject of several studies for many years, the mechanisms underlying their formation, growth and eventual rupture are largely unknown (5). IA are mostly acquired lesions resulting from a defective vascular wall response to local hemodynamic stress (6). The structural deterioration of the arterial wall involves inflammation and tissue degeneration with degradation of the extracellular matrix and smooth muscle cell apoptosis (7). Risk factors such as hypertension, female sex, increasing age, cigarette smoking, excessive alcohol consumption and familial history of aneurysm, predispose to IA formation and rupture (8). Furthermore, increasing evidence suggest a genetic component of IA formation (9). Genome wide association studies and subsequent replication case-control studies have identified common risk alleles for IA formation on chromosomes 4q31-23, 8q11 an 9p21.3 (10). However, these loci explain only 5% of the familial inheritance cases (11).

Whole-exome sequencing approaches have recently been applied to families with multiple IA carriers, leading to the identification of new susceptibility genes for IA pathogenesis, such as RNF213 (12) or THSD1 (13). While the proteinRing Finger Protein 213 had been previously involved in vascular-wall construction (14,15), inactivation of the Thrombospondin Type 1 Domain Containing Protein 1 has been reported to impair the adhesion of endothelial cells to the extracellular matrix, and to cause cerebral bleeding and increased mortality in zebrafish and mice (13). These recent advances provide new insights into the pathophysiology of IA, and demonstrate the usefulness of familial approaches based on next-generation sequencing to improve knowledge on the molecular mechanisms underlying IA formation and rupture.

### SUMMARY OF THE INVENTION

The invention relates to a method for predicting the risk of having or developing Intracranial aneurysms (IA) in a subject, comprising the steps of: i) identifying at least one mutation in an angiogenic protein; and ii) concluding that the subject is at risk of having or developing IA when at least one mutation is identified in an angiogenic protein. In particular, the invention is defined by the claims.

### DETAILED DESCRIPTION OF THE INVENTION

In the present study, inventors have identified one rare nonsense variant (c.1378A>T) in the last exon of the Angiopoietin-Like 6 (ANGPTL6) gene which encodes a circulating pro-angiogenic factor mainly secreted from the liver shared by the 4 tested affected members of a large pedigree with multiple IA carriers. They showed a 50% reduction of ANGPTL6 serum concentration in heterozygous c.1378A>T carriers compared to non-carrier relatives, due to the non-secretion of the truncated protein produced by the c.1378A>T transcripts. Sequencing ANGPTL6 in a series of 94 additional index cases with familial IA identified 3 other rare coding variants in 5 cases. Overall, they detected a significant enrichment (p=0.023) in rare coding variants within this gene among the 95 index cases with familial IA, compared to a reference population of 404 individuals with French ancestry. Among the 6 recruited families, 12 out of 13 (92%) individuals carrying IA also carry such variants in ANGPTL6, versus 15 out of 41 (37%) unaffected ones. They observed a higher rate of individuals with a history of high blood pressure among affected versus healthy carriers of ANGPTL6 variants, suggesting that ANGPTL6 could trigger cerebrovascular lesions when combined with other risk factors such as hypertension. Altogether, their results indicate that rare coding variants in ANGPTL6 are causally related to familial forms of IA.

Method for Predicting the Risk of having or Developing Intracranial Aneurysms

Accordingly, in a first aspect, the present invention relates to a method for predicting the risk of having or developing Intracranial aneurysms (IA) in a subject, comprising the steps of: i) identifying at least one mutation in an angiogenic protein; and ii) concluding that the subject is at risk of having or developing IA when at least one mutation is identified in said angiogenic protein.

More particularly, inventors have identified one rare nonsense variant (c.1378A>T) in the last exon of the Angiopoietin-Like 6 (ANGPTL6) gene, shared by the 4 tested affected members of a large pedigree with multiple IA carriers. Thus, in the context of the invention, the protein angiogenic is Angiopoietin-Like 6 (ANGPTL6).

Accordingly, the present invention relates to a method or predicting the risk of having or developing Intracranial aneurysms (IA) in a subject comprises following steps: i) determining the expression level of ANGPTL6 in a biological sample obtained from said subject, ii) comparing the expression level of ANGPTL6 determined at step i) with a predetermined reference value and iii) concluding that the subject is at risk of having or developing IA when the expression level of ANGPTL6 determined at step i) is lower than the predetermined reference value, or concluding that the patient is not at risk of having or developing IA when the expression level of ANGPTL6 determined at step i) is higher than the predetermined reference value.

As used herein, the term “predicting” means that the subject to be analyzed by the method of the invention is allocated either into the group of subjects who will have or develop IA, or into a group of subjects who will not have or develop IA. Having or developing IA referred to in accordance with the invention, particularly, means that the subject will have higher risk to have or develop IA. Typically, said risk is elevated as compared to the average risk in a cohort of subjects suffering from IA.

In the context of the invention, the risk of having the IA in a subject susceptible to suffer from IA shall be predicted. The term “predicting the risk”, as used herein, refers to assessing the probability according to which the patient as referred to herein will have or develop IA.

As will be understood by those skilled in the art, such an assessment is usually not intended to be correct for 100% of the subjects to be investigated. The term, however, requires that prediction can be made for a statistically significant portion of subjects in a proper and correct manner. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the invention allows that the prediction of an increased risk will be correct for at least 60%, at least 70%, at least 80%), or at least 90% of the subjects of a given cohort or population.

As used herein, the term “Intracranial aneurysms (IA)” also known as brain aneurysm refers to acquired cerebrovascular abnormalities characterized by a localized dilation and wall thinning in intracranial arteries. There are four main types of intracranial aneurysms (IA) as described in Alexander Keedy et al 2006: saccular, fusiform, dissecting, and micotic type. The saccular aneurysm is the most common form of IA. The main IA complication is the rupture, resulting in subarachnoid haemorrhage and possibly leading to severe outcome. Accordingly, the present invention relates also to a method for predicting the risk of having or developing rupture by performing the method as describe above.

As used herein, the term “subject” refers to any mammals, such as a rodent, a feline, a canine, and a primate. Particularly, in the present invention, the subject is a human. In a particular embodiment, the subject is susceptible to have or develop an IA. More particularly, the subject is susceptible to have or develop saccular aneurysms, fusiform aneurysms, microaneurysms or dissecting aneurysms. In particular, the subject suffers from saccular aneurysms.

As used herein, the term “biological sample” refers to a sample obtained from a subject, for example blood, saliva, breast milk, urine, semen, blood plasma, synovial fluid, serum. In particular embodiment, the biological sample is blood sample. More particularly, the biological sample is peripheral blood mononuclear cells (PBMC). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis, which will preferentially lyse red blood cells. Such procedures are known to the experts in the art. Said biological sample is obtained for the purpose of the in vitro evaluation.

As used herein, the term “angiogenic protein” refers to proteins involved in the angiogenesis. Angiogenesis refers to the formation of new blood vessels. Angiogenesis is performed by various angiogenic proteins. In the context of the invention, the angiogenic proteins are selected from the group consisting of but not limited to: FGF, VGF, VEGFR, ANG1, ANG2, PDGF, PDGFR, TGF-beta, TGF-beta receptors, CCL2, histamine, VE-cadherin, β-catenin, p120-catenin, plakoglobin, integrins and Rho proteins. In a particular embodiment, the angiogenic protein is Angiopoietin-Like 6 (ANGPTL6). As used herein, the term “Angiopoietin-Like 6 (ANGPTL6)” also known as angiopoietin-related growth factor (AGF), is a secreted 50 kDa protein that contains a coiled-coil domain and a fibrinogen-like domain. The naturally occurring human ANGPTL6 gene has a nucleotide sequence as shown in Genbank Accession numbers NM_001321411.1 1 and NM_031917.2, and the naturally occurring human TCL1A protein has an aminoacid sequence as shown in Genbank Accession numbers NP_001308340.1 and NP_114123.2.

As used herein, the term “gene” has its general meaning in the art and refers to means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed.

As used herein the “allele” has its general meaning in the art and refers to an alternative form of a gene (one member of a pair) that is located at a specific position on a specific chromosome which, when translated result in functional or dysfunctional (including non-existent) gene products.

As used herein, the term “mutation” has its general meaning in the art and refers to any detectable change in genetic material, e.g. DNA, RNA, cDNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. Mutations include deletion, insertion or substitution of one or more nucleotides. The mutation may occur in the coding region of a gene (i.e. in exons), in introns, or in the regulatory regions (e.g. enhancers, response elements, suppressors, signal sequences, polyadenylation sequences, promoters) of the gene. Generally a mutation is identified in a subject by comparing the sequence of a nucleic acid or polypeptide expressed by said subject with the corresponding nucleic acid or polypeptide expressed in a control population. Where the mutation is within the gene coding sequence, the mutation may be a “mis sense” mutation, where it replaces one amino acid with another in the gene product, or a “non sense” mutation, where it replaces an amino acid codon with a stop codon. A mutation may also occur in a splicing site where it creates or destroys signals for exon-intron splicing and thereby lead to a gene product of altered structure. A mutation in the genetic material may also be “silent”, i.e. the mutation does not result in an alteration of the amino acid sequence of the expression product.

As used herein, the term “homozygous” refers to an individual possessing two copies of the same allele. As used herein, the term “homozygous mutant” refers to an individual possessing two copies of the same allele, such allele being characterized as the mutant form of a gene.

As used herein, the term “heterozygous” refers to an individual possessing two different alleles of the same gene, i.e. an individual possessing two different copies of an allele, such alleles are characterized as mutant forms of a gene.

In a particular embodiment, the mutation allows to a truncated protein. Typically, truncated protein refers to a protein shortened by a mutation which specifically induces premature termination of messenger RNA translation.

In the context of the invention, inventors have identified one rare nonsense variant, c.1378A>T, in the last exon of the Angiopoietin-Like 6 (ANGPTL6) gene, which was carried by the 4 affected members from a large pedigree with multiple IA carriers, and by only 5 out of 22 unaffected relatives. The result suggests that this variant in ANGPLT6 may be the major gene susceptibility factor for IA in this family with multiple carriers.

Inventors have also identified a truncated form of ANGPTL6 which lacks the last 11 C-terminal residues. Typically, K460 in the human Angptl6 sequence corresponds to K447 in the mouse sequence and the K447*Angplt6 mouse mutant also lacks the last 11 C-terminal residues.

Accordingly, the present invention also relates to a method for predicting the risk of having or developing intracranial aneurysms (IA) in a subject in need thereof, comprising the step of detecting angiogenic protein (e.g ANGPTL6) single nucleotide polymorphism (SNP) in a biological sample obtained from said subject.

In a further aspect, the present invention relates to a method for predicting the risk of having or developing intracranial aneurysms (IA) in a subject in need thereof, comprising the step of determining the expression level of ANGPTL6 and/or detecting ANGPTL6 SNP in a biological sample obtained from said subject.

In a particular embodiment, the invention relates to a method for predicting the risk of having or developing Intracranial aneurysms (IA) in a subject in need thereof, comprising the steps of: i) determining the expression level of angiogenic protein (e.g. ANGPTL6) and/or detecting angiogenic protein SNP (e.g. ANGPTL6 SNP) in a biological sample obtained from said subject, ii) comparing the expression level determined at step i) with a predetermined reference value and iii) concluding that the subject is at risk of having or developing IA when the expression level determined at step i) is lower than the predetermined reference value and/or when the angiogenic protein SNP (e.g. ANGPTL6 SNP) is detected, or concluding that the patient is not at risk of having or developing IA when the expression level determined at step i) is higher than the predetermined reference value and/or when the SNP is not detected.

In a particular embodiment, the mutation is a nonsense variant such as c.1378A>T.

In another embodiment, the mutation leads to a truncated protein, such as a mutation in K460 in the human ANGPTL6.

As used herein, the term “single nucleotide polymorphism (SNP)” refers to is a single basepair variation in a nucleic acid sequence of an angiogenic (e.g.ANGPTL6) gene. Polymorphisms can be referred to, for instance, by the nucleotide position at which the variation exists, by the change in amino acid sequence caused by the nucleotide variation, or by a change in some other characteristic of the nucleic acid molecule that is linked to the variation {e.g., an alteration of a secondary structure such as a stem-loop, or an alteration of the binding affinity of the nucleic acid for associated molecules, such as polymerases, RNases, and so forth). For example, the SNP in the context of the invention is mis sense mutation in exon 1 leading to the E131V substitution in ANGPTL6, a missense mutation in exon 4 leading to the L348F substitution, or CGCGCTGAGCCTCGGCGGA-bp (SEQ ID NO: 1) insertion leading to one premature STOP codon in exon 2.

In the methods according to the present the invention, the presence or absence of a SNP can be determined by nucleic acid sequencing, PCR analysis or any genotyping method known in the art such as the method described in the example. Examples of such methods include, but are not limited to, chemical assays such as allele specific hybridization (DASH), pyrosequencing, molecular beacons, SNP microarrays, restriction fragment length polymorphism (RFLP), flap endonuclease (FEN), single strand conformation polymorphism, temperature gradient gel electrophoresis (TGGE), denaturing high performance liquid chromatography (DHPLC), high-resolution melting of the entire amplicon, and DNA mismatch-binding proteins. primer extension, allele specific oligonucleotide ligation, sequencing, enzymatic cleavage, flap endonuclease discrimination; and detection methods such as fluorescence, chemiluminescence, and mass spectrometry.

For example, the presence or absence of said polymorphism may be detected in a DNA sample, preferably after amplification. For instance, the isolated DNA may be subjected to couple reverse transcription and amplification, such as reverse transcription and amplification by polymerase chain reaction (RT-PCR), using specific oligonucleotide primers that are specific for the polymorphism or that enable amplification of a region containing the polymorphism. According to a first alternative, conditions for primer annealing may be chosen to ensure specific reverse transcription (where appropriate) and amplification; so that the appearance of an amplification product be a diagnostic of the presence of the polymorphism according to the invention. Otherwise, DNA may be amplified, after which a mutated site may be detected in the amplified sequence by hybridization with a suitable probe or by direct sequencing, or any other appropriate method known in the art.

Currently numerous strategies for genotype analysis are available (Antonarakis et al., 1989; Cooper et al., 1991; Grompe, 1993). Briefly, the nucleic acid molecule may be tested for the presence or absence of a restriction site. When a base polymorphism creates or abolishes the recognition site of a restriction enzyme, this allows a simple direct PCR genotype the polymorphism. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; mass spectrometry sequencing; pyrosequencing; sequencing using a chip-based technology and real-time quantitative PCR. Preferably, DNA from a patient is first subjected to amplification by polymerase chain reaction (PCR) using specific amplification primers. However several other methods are available, allowing DNA to be studied independently of PCR, such as the rolling circle amplification (RCA), the Invader™ assay, or oligonucleotide ligation assay (OLA). OLA may be used for revealing base polymorphisms. According to this method, two oligonucleotides are constructed that hybridize to adjacent sequences in the target nucleic acid, with the join sited at the position of the polymorphism. DNA ligase will covalently join the two oligonucleotides only if they are perfectly hybridized to one of the allele.

Oligonucleotide probes or primers may contain at least 10, 15, 20 or 30 nucleotides. Their length may be shorter than 400, 300, 200 or 100 nucleotides.

According to the invention, the determination of the presence or absence of said SNP may also be determined by detection or not of the mutated protein by any method known in the art. The presence of the protein of interest may be detected using standard electrophoretic and immunodiagnostic techniques, including immunoassays such as competition, direct reaction, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith. Labels are known in the art that generally provide (either directly or indirectly) a signal. As used herein, the term “labelled” with regard to the antibody or aptamer, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or indocyanine (Cy5), to the antibody or aptamer, as well as indirect labelling of the probe or antibody (e.g., horseradish peroxidise, HRP) by reactivity with a detectable substance. An antibody or aptamer may be also labelled with a radioactive molecule by any method known in the art. For example, radioactive molecules include but are not limited radioactive atom for scintigraphic studies such as I123, I124, In111, Re186 and Re188. The aforementioned assays generally involve separation of unbound protein in a liquid phase from a solid phase support to which antigen-antibody complexes are bound. Solid supports which may be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, etc.

More particularly, an ELISA method may be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

Alternatively, an immunohistochemistry (IHC) method may be used. IHC specifically provides a method of detecting a target in a biological sample or tissue specimen in situ. The overall cellular integrity of the sample is maintained in IHC, thus allowing detection of both the presence and location of the target of interest. Typically a biological sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan).

In one embodiment of the present invention, direct sequencing of the whole genome is used to detect the SNP locus ANGPTL6. The whole genome sequencing may be achieved by use of the next generation sequencing (NGS) assay. In NGS, a single genomic DNA is first fragmented into a library of small segments that can be uniformly and accurately sequenced in millions of parallel reactions. The newly identified strings of bases, called reads, are then reassembled using a known reference genome as a scaffold (resequencing), or in the absence of a reference genome (de novo sequencing). The full set of aligned reads would reveal the entire sequence of each chromosome of the genomic DNA.

In another embodiment of the present invention, primer extension assay is used to detect the SNP locus ANGPTL6. The primer extension assay may be achieved by use of Matrix assisted laser desorption ionization time-of-flight mass spectrometry (MALDI-TOF MS). Mass spectrometry is an experimental technique used to identify the components of a heterogeneous collection of biomolecules, by sensitive discrimination of their molecular masses. In MALTI-TOF MS, the sample to be analyzed is placed in a UV-absorbing matrix pad and exposed to a short laser pulse. The ionized molecules are accelerated off the matrix pad (i.e., desorption) and move into an electric field towards a detector. The “time of flight” required to reach the detector depends on the mass/charge (m/z) ratio of the individual molecules. To use MALTI-TOF MS for DNA sequencing, the DNA sequence to be sampled is first transcribed into RNA in vitro in 4 separate reactions, each with three rNTP bases and one specific dNTP. The incorporated dNTP in the transcribed RNA will prevent cleavage from occurring at that dNTP position by RNAse, and therefore generate distinct fragments. Each fragment has a characteristic m/z ratio that appears as a peak in MALTI-TOF spectrum. The MALTI-TOF mass signal pattern obtained for the DNA sample is then compared with the expected m/z spectrum of the reference sequence, which includes the products of all 4 cleavage reactions. Any SNP differences between the sample DNA and the reference DNA sequences will produce predictable shifts in the spectrum, and their exact nature can be deduced.

In still another embodiment of the present invention, quantitative polymerase chain reaction (qPCR) is used to detect the desired SNP locus. In qPCR, DNA sample that includes the SNP locus is amplified and simultaneously detected and quantitated with different primer sets that target each allele separately. Well-designed primers will amplify their target SNP at a much earlier cycle than the other SNPs. This allows more than two alleles to be distinguished, although an individual qPCR reaction is required for each SNP. To achieve high enough specificity, the primer sequence may require placement of an artificial mismatch near its 3′-end, which is an approach generally known as Taq-MAMA. This artificial mismatch induces a much greater amplification delay for non-target alleles than a single mismatch would alone, yet does not substantially affect amplification of the target SNP.

In still another embodiment of the present invention, the SNP locus is detected by direct sequencing of a specified DNA segment containing the SNP locus of ANGPTL6. As used herein, the term “expression level” refers to the expression level of angiogenic gene with further other values corresponding to the clinical parameters. Typically, the expression level of the gene may be determined by any technology known by a person skilled in the art. In particular, each gene expression level may be measured at the genomic and/or nucleic and/or protein level. In a particular embodiment, the expression level of ANGPTL6 gene is measured. The expression level of ANGPTL6 is assessed by analyzing the expression of the protein translated from said gene. Said analysis can be assessed using an antibody (e.g., a radio-labelled, chromophore-labelled, fluorophore-labelled, or enzyme-labelled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for ANGPTL6.

Methods for measuring the expression level of an angiogenic protein, particularly, ANGPTL6 in a sample may be assessed by any of a wide variety of well-known methods from one of skill in the art for detecting expression of a protein including, but not limited to, direct methods like mass spectrometry-based quantification methods, protein microarray methods, enzyme immunoassay (EIA), radioimmunoassay (RIA), Immunohistochemistry (IHC), Western blot analysis, ELISA, Luminex, ELISPOT and enzyme linked immunosorbent assay and indirect methods based on detecting expression of corresponding messenger ribonucleic acids (mRNAs). The mRNA expression profile may be determined by any technology known by a man skilled in the art. In particular, each mRNA expression level may be measured using any technology known by a man skilled in the art, including nucleic microarrays, quantitative Polymerase Chain Reaction (qPCR), next generation sequencing and hybridization with a labelled probe.

Said direct analysis can be assessed by contacting the sample with a binding partner capable of selectively interacting with the biomarker present in the sample. The binding partner may be an antibody that may be polyclonal or monoclonal, preferably monoclonal (e.g., a isotope-label, element-label, radio-labelled, chromophore-labelled, fluorophore-labelled, or enzyme-labelled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for the biomarker of the invention. In another embodiment, the binding partner may be an aptamer.

The binding partners of the invention such as antibodies or aptamers, may be labelled with a detectable molecule or substance, such as an isotope, an element, a fluorescent molecule, a radioactive molecule or any others labels known in the art. Labels are known in the art that generally provide (either directly or indirectly) a signal.

As used herein, the term “labelled”, with regard to the antibody, is intended to encompass direct labelling of the antibody or aptamer by coupling (i.e., physically linking) a detectable substance, such as an isotope, an element, a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be produced with a specific isotope or a radioactive molecule by any method known in the art. For example radioactive molecules include but are not limited to radioactive atom for scintigraphy studies such as I123, I124, In111, Re186, Re188, specific isotopes include but are not limited to 13C, 15N, 126I, 79Br, 81Br.

The aforementioned assays generally involve the binding of the binding partner (ie. antibody or aptamer) to a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidene fluoride; diazotized paper; nylon membranes; activated beads, magnetically responsive beads, silicon wafers.

In a particular embodiment, an ELISA method can be used, wherein the wells of a microtiter plate are coated with a set of antibodies which recognize angiogenic protein, such as ANGPTL6. A sample containing or suspected of containing said biomarker is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art such as Singulex, Quanterix, MSD, Bioscale, Cytof.

In one embodiment, an Enzyme-linked immunospot (ELISpot) method may be used. Typically, the sample is transferred to a plate which has been coated with the desired anti-angiogenic protein (e.g.ANGPTL6) capture antibodies. Revelation is carried out with biotinylated secondary Abs and standard colorimetric or fluorimetric detection methods such as streptavidin-alkaline phosphatase and NBT-BCIP and the spots counted.

In one embodiment, when multi-biomarker expression measurement is required, use of beads bearing binding partners of interest may be preferred. In a particular embodiment, the bead may be a cytometric bead for use in flow cytometry. Such beads may for example correspond to BD™ Cytometric Beads commercialized by BD Biosciences (San Jose, California). Typically cytometric beads may be suitable for preparing a multiplexed bead assay. A multiplexed bead assay, such as, for example, the BD™ Cytometric Bead Array, is a series of spectrally discrete beads that can be used to capture and quantify soluble antigens. Typically, beads are labelled with one or more spectrally distinct fluorescent dyes, and detection is carried out using a multiplicity of photodetectors, one for each distinct dye to be detected. A number of methods of making and using sets of distinguishable beads have been described in the literature. These include beads distinguishable by size, wherein each size bead is coated with a different target-specific antibody (see e.g. Fulwyler and McHugh, 1990, Methods in Cell Biology 33:613-629), beads with two or more fluorescent dyes at varying concentrations, wherein the beads are identified by the levels of fluorescence dyes (see e.g. European Patent No. 0 126,450), and beads distinguishably labelled with two different dyes, wherein the beads are identified by separately measuring the fluorescence intensity of each of the dyes (see e.g. U.S. Pat. Nos. 4,499,052 and 4,717,655). Both one-dimensional and two-dimensional arrays for the simultaneous analysis of multiple antigens by flow cytometry are available commercially. Examples of one-dimensional arrays of singly dyed beads distinguishable by the level of fluorescence intensity include the BD™ Cytometric Bead Array (CBA) (BD Biosciences, San Jose, Calif.) and Cyto-Plex™ Flow Cytometry microspheres (Duke Scientific, Palo Alto, Calif.). An example of a two-dimensional array of beads distinguishable by a combination of fluorescence intensity (five levels) and size (two sizes) is the QuantumPlex™ microspheres (Bangs Laboratories, Fisher, Ind.). An example of a two-dimensional array of doubly-dyed beads distinguishable by the levels of fluorescence of each of the two dyes is described in Fulton et al. (1997, Clinical Chemistry 43(9):1749-1756). The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange). In another particular embodiment, bead is a magnetic bead for use in magnetic separation. Magnetic beads are known to those of skill in the art. Typically, the magnetic bead is preferably made of a magnetic material selected from the group consisting of metals (e.g. ferrum, cobalt and nickel), an alloy thereof and an oxide thereof. In another particular embodiment, bead is bead that is dyed and magnetized.

In one embodiment, protein microarray methods may be used. Typically, at least one antibody or aptamer directed against an angiogenic protein (e.g.ANGPTL6) is immobilized or grafted to an array(s), a solid or semi-solid surface(s). A sample containing or suspected of containing an angiogenic protein (e.g.ANGPTL6) is then labelled with at least one isotope or one element or one fluorophore or one colorimetric tag that are not naturally contained in the tested sample. After a period of incubation of said sample with the array sufficient to allow the formation of antibody-antigen complexes, the array is then washed and dried. After all, quantifying an angiogenic protein (e.g.ANGPTL6) may be achieved by using any appropriate microarray scanner like fluorescence scanner, colorimetric scanner, SIMS (secondary ions mass spectrometry) scanner, maldi scanner, electromagnetic scanner or any technique allowing to quantify said labels.

In another embodiment, the antibody or aptamer grafted on the array is labelled.

In another embodiment, reverse phase arrays may be used. Typically, at least one sample is immobilized or grafted to an array(s), a solid or semi-solid surface(s). An antibody or aptamer against the suspected biomarker is then labelled with at least one isotope or one element or one fluorophore or one colorimetric tag that are not naturally contained in the tested sample. After a period of incubation of said antibody or aptamer with the array sufficient to allow the formation of antibody-antigen complexes, the array is then washed and dried. After all, detecting quantifying and counting by D-SIMS said biomarker containing said isotope or group of isotopes, and a reference natural element, and then calculating the isotopic ratio between the biomarker and the reference natural element. may be achieve using any appropriate microarray scanner like fluorescence scanner, colorimetric scanner, SIMS (secondary ions mass spectrometry) scanner, maldi scanner, electromagnetic scanner or any technique allowing to quantify said labels.

In one embodiment, said direct analysis can also be assessed by mass Spectrometry. Mass spectrometry-based quantification methods may be performed using either labelled or unlabelled approaches (DeSouza and Siu, 2012). Mass spectrometry-based quantification methods may be performed using chemical labeling, metabolic labelingor proteolytic labeling. Mass spectrometry-based quantification methods may be performed using mass spectrometry label free quantification, LTQ Orbitrap Velos, LTQ-MS/MS, a quantification based on extracted ion chromatogram EIC (progenesis LC-MS, Liquid chromatography-mass spectrometry) and then profile alignment to determine differential expression of the biomarker.

In another embodiment, the angiogenic protein, particularly ANGPTL6 expression level is assessed by analyzing the expression of mRNA transcript or mRNA precursors, such as nascent RNA, of ANGPTL6 gene. Said analysis can be assessed by preparing mRNA/cDNA from cells in a sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFFYMETRIX).

Advantageously, the analysis of the expression level of mRNA transcribed from the gene encoding for biomarkers involves the process of nucleic acid amplification, e. g., by RT-PCR (the experimental embodiment set forth in U.S. Pat. No. 4,683,202), ligase chain reaction (Barany, 1991), self-sustained sequence replication (Guatelli et al., 1990), transcriptional amplification system (Kwoh et al., 1989), Q-Beta Replicase (Lizardi et al., 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

As used herein, the term “predetermined reference value” refers to a threshold value or a cut-off value. The setting of a single “reference value” thus allows discrimination between a subject at risk of having or developing IA and a subject not at risk of having or developing IA with respect to the overall survival (OS) for a subject. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the expression level (obtained according to the method of the invention) with a defined threshold value. In one embodiment of the present invention, the threshold value is derived from the expression level (or ratio, or score) determined in a biological sample derived from one or more subjects at risk of having or developing IA. Furthermore, retrospective measurement of the expression level (or ratio, or scores) in properly banked historical subject samples may be used in establishing these threshold values.

Predetermined reference values used for comparison may comprise “cut-off” or “threshold” values that may be determined as described herein. Each reference (“cut-off”) value for ANGPTL6 may be predetermined by carrying out a method comprising the steps of

a) providing a collection of samples from subjects at risk of having or developing IA;

b) determining the expression level of angiogenic protein for each sample contained in the collection provided at step a);

c) ranking the biological samples according to said expression level;

d) classifying said samples in pairs of subsets of increasing, respectively decreasing, number of members ranked according to their expression level,

e) providing, for each sample provided at step a), information relating to the risk of having or developing IA or the actual clinical outcome for the corresponding subject (i.e. the duration of the overall survival (OS));

f) for each pair of subsets of samples, obtaining a Kaplan Meier percentage of survival curve;

g) for each pair of subsets of samples calculating the statistical significance (p value) between both subsets;

h) selecting as reference value for the expression level, the value of expression level for which the p value is the smallest.

For example the expression level of angiogenic protein (e.g. ANGPTL6) has been assessed for 100 samples of 100 patients. The 100 samples are ranked according to their expression level. Sample 1 has the best expression level and sample 100 has the worst expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

In routine work, the reference value (cut-off value) may be used in the present method to discriminate samples and therefore the corresponding patients.

Kaplan-Meier curves of percentage of survival as a function of time are commonly to measure the fraction of patients living for a certain amount of time after treatment and are well known by the man skilled in the art.

The man skilled in the art also understands that the same technique of assessment of the expression level of angiogenice protein (e.g. ANGPTL) should of course be used for obtaining the reference value and thereafter for assessment of the expression level of a biomarker of a patient subjected to the method of the invention.

In one embodiment, the reference value may correspond to the expression level of angiogenice protein (e.g. ANGPTL6) determined in a sample associated with subject at risk of having or developing IA. Accordingly, a lower expression level of angiogenic protein than the reference value is indicative of a subject at risk of having or developing IA, and a higher or equal expression level of angiogenic protein than the reference value is indicative of a subject not at risk of having or developing acute IA.

In another embodiment, the reference value may correspond to the expression level of angiogenic protein (e.g.ANGPTL6) determined in a sample associated with subject not at risk of having or developing IA. Accordingly, a higher or equal expression level of ANGPTL6 (e.g.ANGPTL6) than the reference value is indicative of a subject not at risk of having or developing IA, and a lower expression level of an angiogenic protein (e.g.ANGPTL6) than the reference value is indicative of a subject at risk of having or developing IA.

### Method for Treating and/or Preventing Intracranial Aneurysms

In a third aspect, the present invention relates to a method of treating and/or preventing intracranial aneurysms in a subject, wherein the subject has been diagnosed as at risk of having or developing intracranial aneurysms (IA) by performing the method of the invention. The method according to the invention, wherein, the treatment and/or prevention consists but not limited to surgery and endovascular techniques. The surgical management of cerebral aneurysms, in which a clip is placed across the neck of the aneurysm, is an effective and safe procedure with the evolution of microsurgical techniques in the hands of an experienced surgeon. Endovascular techniques can be divided into: parent artery reconstruction with coil deposition (primary coil, balloon-assisted coiling, stent-assisted coiling, and other new techniques such as neck reconstruction devices and intraluminal occlusion devices); reconstruction with flow diversion; and deconstructive techniques with involving parent artery sacrifice with or without bypass.

Thus, surgery and endovascular techniques for use in a method of treating and/or preventing intracranial aneurysms in a subject in need thereof, wherein the subject has been diagnosed as at risk of having or developing intracranial aneurysms IA by performing the method of the invention.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

### Kit

In a fourth aspect, the invention relates to a kit for performing the methods of the present invention, wherein said kit comprises means for measuring the expression level of an angiogenic protein (e.g. ANGPTL6) and/or detecting angiogenic SNP (e.g.ANGPTL6 SNP) that is indicative of subject at risk of having or developing Intracranial aneurysms (IA).

Typically the kit may include antibodies, primers, probes, macroarrays or microarrays as above described. For example, the kit may comprise a set of antibodies, primers, or probes as above defined, and optionally pre-labelled. Alternatively, antibodies, primers, or probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. The kit may further comprise amplification reagents and also other suitably packaged reagents and materials needed for the particular amplification protocol.

The invention will be further illustrated by the following figures and examples. However, these examples and figures should not be interpreted in any way as limiting the scope of the present invention.

### FIGURES

FIG. 1: Genetic investigations in a large family with multiple IA carriers Pedigree of family A showing the segregation pattern of the variant ANGPTL6 c.1378A>T (Filled, empty boxes and boxes with question marks indicate IA carriers, non-carriers and individuals with unknown status; signs ‘+’ indicate the presence of the ANGPTL6 variant, signs ‘−’ its absence; the arrow indicates the index case, the asterisks indicate the individuals included in WES analysis).

FIG. 2: Expression of WT- and Lys460Ter-Lys460Ter-ANGPTL6 in cultured cells and individual sera

(A) Analysis by qPCR of ANGPTL6 transcripts in HEK293 cells expressing WT- and Lys460Ter-ANGPTL6. (B) Analysis of serum level of ANGPTL6 in controls (WT-ANGPTL6) and individuals expressing the Lys460Ter-ANGPTL6 (heterozygous) (**P<0.01).

FIG. 3: Familial cases of IA in the presence of rare coding variants in ANGPTL6

Filled, empty boxes and boxes with question marks indicate IA carriers, non-carriers and individuals with unknown status; signs ‘+’ indicate the presence of the ANGPTL6 variant, signs ‘−’ its absence; black arrows indicates the index cases.

FIG. 4: Angptl6 mutant mice display dilated arteries under basal condition. The passive diameter of isolated basilar artery pressured at 50 mm Hg is significantly larger in Angptl6+/Δ. and Angptl6/Δ. mice than in controls.

FIG. 5: Cerebral arteries of Angptl6 mutant mice abnormally dilate in high blood pressure condition. Hypertension did not modify the diameter of the basilar artery in control Angptl6+/+ mice (red dashed line) while it induces an increase of this diameter in Angptl6+/Δ and Angptl6Δ/Δ. mice (dotted line). The difference in the arterial diameter of Angptl6+/+ mice and that of Angptl6+/Δ and Angptl6Δ/Δ mice is thus potentiated in high blood pressure condition (*P<0.05; **P<0.01).

FIG. 6: Change in mechanical properties of isolated basilar artery from Angptl6 mutant mice. Flow-mediated dilation, corresponding the increase in the diameter of the artery in response to a gradual increase in the intraluminal flow from 3 to 10 μl/min is reduced in Angptl6Δ/Δ mice compared with Angptl6+/+ mice. After L-NAME treatment to inhibit endothelial NO synthesis, flow-mediated dilation is similar in arteries from Angptl6+/+ and from Angptl6Δ/Δ mice. This indicates that a reduced production of NO in response to flow in Angptl6/Δ mice. (*P<0.05; **P0.01).

### EXAMPLES

### Example 1

Material & Methods

Clinical Recruitment

Familial cases of IA are defined as at least two first-degree relatives both diagnosed with typical IA (defined as a saccular arterial dilatation of any size occurring at a bifurcation of the intracranial vasculature), without any age limitation. Index cases and their relatives were recruited following the French ethical guidelines for genetic research, and under approval from the French Ministry of Research (n° DC-2011-1399) and the local ethical committee. Informed written consent was obtained from each individual agreeing to participate in the genetic study, to whom MRI screening and blood sampling were proposed.

The full recruiting process has been described previously (16). Briefly, neuroradiological phenotyping was performed in each recruiting center by interventional neuroradiologists, neurologists and neurosurgeons in order to recruit only cases with typical saccular bifurcation IA. Mycotic, fusiform-shaped or dissecting IA were systematically excluded, as well as IA in relation with an arteriovenous malformation and IA resulting from syndromic disorders such as Marfan disease or vascular forms of Elhers Danlos. Eye fundus, transthoracic echocardiography, non-invasive analysis of endothelial dysfunction, and Doppler echography analysis of peripheral arteries (sub clavians, radials, femorals, renals, and digestives) were carried out to check for any other vascular malformation or variation potentially linked to the presence of IA, thus constituting a syndrome yet unknown.

Whole Exome Sequencing (WES)

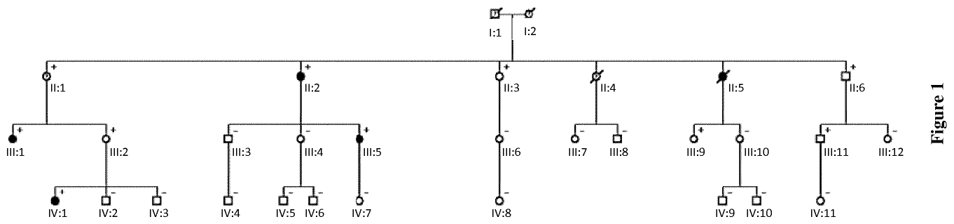

Genomic DNA was extracted from peripheral blood lymphocytes using the NucleoSpin® Blood kit XL (Macherey Nagel, Germany). Briefly, coding exons from 3 μg of genomic DNA were captured using the SureSelect Human All Exon V4 Kit (Agilent Technologies, Santa Clara, Calif.), following the manufacturer's protocol. DNA was sheared by acoustic fragmentation (Bioruptor Diagenode) and purified with the magnetic beads Agencourt AMPure XP (Beckmann Coulter genomics), and fragment quality was assessed (Tapestation 2200 Agilent). Exome-enriched genomes were paired-end sequenced (100-bp reads) on IIlumina HiSeq 1500 (Illumina Inc, San Diego, Calif.) to a mean depth above 30×. Sequence reads were mapped to the human reference genome (Broad Institute human_glk_v37) using the Burrows-Wheeler Aligner (17). Duplicates were flagged using Picard software. Reads were realigned and recalibrated using the Genome Analysis Toolkit (GATK) (18). Variant detection was performed with GATK HaplotypeCaller. Functional annotation of high-quality variants was performed using Ensembl VEPv7.4. The sequencing quality was determined with the Depth Of Coverage Walker provided in GATK. Knime4Bio (19) was used for all merging and filtering steps. Variants with a sequencing depth of less than 10 or a genotype quality below 90 were excluded, as well as synonymous variants with no predicted effect on splicing sites. At last, from the resulting set of ‘functional’ variants (as reported in FIG. 1), we filtered out any variant with a minor allele frequency (MAF) higher than 0.1% in the non-Finnish European (NFE) population from the ExAC database, as well as few remaining variants reported with a minor allele frequency (MAF) higher than 10% in our in-house database of 260 whole-exome sequences from individuals with various cardiac phenotypes.

Identity-by-Descent Analysis

SNP genotyping was performed on population-optimized Affymetrix Axiom Genome-Wide CEU 1 array plates following the standard manufacturer's protocol. Fluorescence intensities were quantified using the Affymetrix GeneTitan Multi-Channel Instrument, and primary analysis was conducted with Affymetrix Power Tools following the manufacturer's recommendations. After genotype calling, all individuals had a genotype call rate above 97%. SNPs with an MAF <10%, a call rate <95% or with P<1×10-5 when testing for Hardy-Weinberg equilibrium were excluded. IBD estimation was performed with IBDLD v3.34, NoLD method (20). Shared regions were obtained by analyzing a set of independent SNPs (R²<0.2) using genotypes from French individuals (21) as a reference panel. The IBD status at every SNP locus was obtained for each pair of individuals, based on a hidden Markov model implemented in the IBDLD program. Still using IBDLD, we estimated the kinship coefficients between pairs of IA cases from distinct pedigrees. We invariably found values around 0.025, thus excluding non-documented close relatedness between mutation carriers.

Capillary Sequencing and Burden Testing

Validation experiments for each selected variant, familial segregation analyses and further screening for ANGPTL6 coding mutations were performed by capillary sequencing on an Applied Biosystems 3730 DNA Analyzer, using standard procedures. Sequences analyses were performed with SeqScape v2.5. ANGPTL6 variants were numbered according to the canonical transcript (ENST00000253109/NM_031917, protein accession number: ENSP00000253109/NP_114123). Burden test was performed using SKAT (22) and CAST (23), by comparing the proportion of individuals carrying at least one rare coding variant within ANGPTL6 (defined as a variant with an MAF below 1% among the 7,509 whole-genome sequenced individuals with NFE ancestry from the gnomAD database) among IA cases versus healthy individuals with French ancestry. ANGPTL6 status in control individuals was determined by whole-genome sequencing with a mean depth of coverage above 30×. Rare variants were defined as variants with an MAF below 1% among the 7,509 whole-genome sequenced individuals with NFE ancestry from the gnomAD database. The count of alleles with rare coding variants in ANGPTL6 among cases was also compared with the same allele count among the 7,509 whole-genome sequenced individuals with NFE ancestry from gnomAD (24), through the use of Fisher's exact test.

Expression Analyses of ANGPTL6

HEK293 cells were maintained in Dulbecco's modified Eagle's medium (DMEM) supplemented with 10% fetal bovine serum and 1% penicillin-streptomycin. Stable HEK293 cell lines were obtained by transfection of pcDNA3.1 vector encoding WT-ANGPTL6 and Lys460Ter-ANGPTL6 (G418 selection). Recombinant proteins are expressed as Nter-FLAG fusion proteins. In HEK293, recombinant human proteins were detected by both anti-flag and anti-ANGPTL6 antibodies (Adipogen, AB_2490340) and ELISA (kit supplied by Adipogen).

In human subjects, serum ANGPTL6 levels were measured by ELISA. For transcript analysis, total RNA from stably transfected HEK293 cells was purified using Trizol (Life technology) according to the manufacturer's instructions then reverse-transcribed. Real-time quantitative PCR was performed using the TaqMan 7900 Sequence Detection System (Applied Biosystems). Primers used to assess ANGPTL6 mRNA expression were designed using the Primer Express 3.1 software (sequences available on request).

Results

A Nonsense Variant in ANGPTL6 Shared by Family Members with IA

The index case of family A (individual III-1; FIG. 1) was diagnosed after a subarachnoid hemorrhage (SAH) at the age of 51. This event revealed a ruptured anterior cerebral artery aneurysm and a second middle cerebral artery aneurysm (data not shown). She completely recovered from the subarachnoid hemorrhage and because of known familial history of ruptured IA (11-2, 11-5), a systematic screening was performed among relatives. Her cousin (III-5) and her niece (IV-1) were both diagnosed with respectively two and one IA. Her uncle (II-4) had an episode suggestive of aneurysmal SAH at the age of 36, and died before a CT scan or angiography could be performed. Her mother (II-1), who carries an ectasia measuring less than 2mm and diagnosed as uncertain (16), was classified as phenotype unknown.

Clinical information was collected for 28 individuals from family A (data not shown). IA was diagnosed on CT angiography or conventional angiography. DNA was available for 27 of them (DNA was unavailable for II-5 who died in 1974 after a rupture of IA). Individuals with IA (II-2, II-5, III-1, III-5 and IV-1) were all female. Noteworthy, all IA carriers except IV-1 suffered from high blood pressure.

We combined WES and IBD analysis to identify any rare genetic variant likely explaining this familial form of IA. Whole-exome sequencing applied to the first cousins III-1 and II-5 led respectively to the detection of 25,674 and 23,456 functional sequence variants in comparison to the human reference genome assembly (data not shown). After filtering out genetic variants reported with an MAF above 0.1% in the non-Finnish European (NFE) population from the ExAC database (24), we ended up with 29 rare variants shared between the first cousins, which were all manually reviewed by visual inspection of sequence reads using the Integrative Genomics Viewer (25).

In parallel, IBD analysis of the complete pedigree identified 12 haplotypes shared by the 4 affected relatives. Within these chromosomal intervals, individuals III.1 and III.5 shared 10 rare, non-synonymous variants (data not shown). By capillary sequencing, we determined that the 4 affected relatives share 8 of these variants (data not shown), among which one nonsense variant, c.1378A>T (p.Lys460Ter), in the ANGPTL6 gene.

Reduced ANGPTL6 Secretion Among Heterozygous Carriers

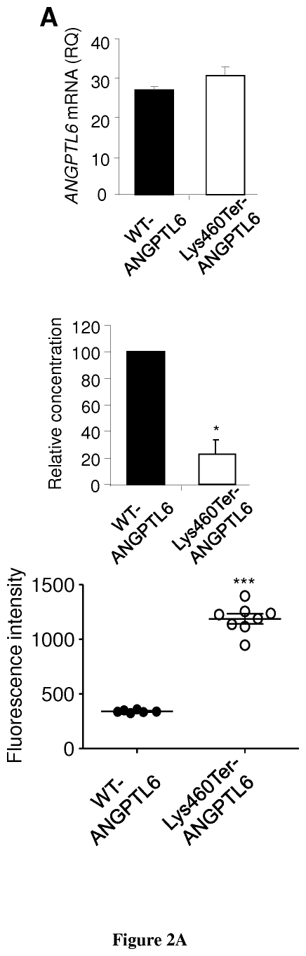

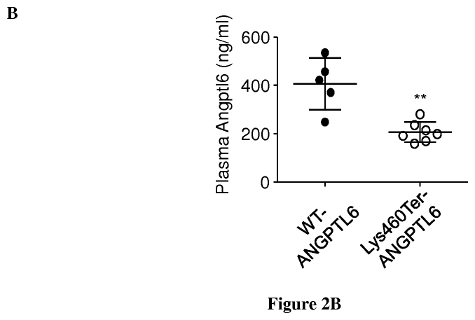

ANGPTL6 is one of the eight members of the secreted glycoprotein ANGPTL family, which share a common structure consisting of an amino-terminal coiled-coil domain, a linker region and a carboxy-terminal fibrinogen-like domain. The c.1378A>T ANGPTL6 variant leads to the occurrence of a premature stop codon in the last exon. The corresponding transcript may thus escape the nonsense-mediated mRNA decay and is predicted to result in a protein truncated by the last 11 amino acids (Lys460Ter-ANGPTL6). To analyze the functional properties of Lys460Ter-ANGPTL6, we established stable cell lines expressing similar levels of the wild-type (WT-ANGPTL6) and mutated (Lys460Ter-ANGPTL6) transcripts, respectively (FIG. 2A). Western blot using anti-flag antibody showed that WT-ANGPTL6 was secreted in the culture medium while Lys460Ter-ANGPTL6 was almost not detected in the supernatant of cells transfected with the variant (FIG. 2A). Quantification of ANGPTL6 concentration by ELISA confirmed the significant reduction of the secretion of Lys460Ter-ANGPTL6 compared to WT-ANGPTL6 (FIG. 2B). In agreement with this defective secretion, immunofluorescence labeling and quantification in permeabilized cells clearly showed the retention of Lys460Ter-ANGPTL6 in the cytoplasm (FIG. 2A). Altogether, these data strongly suggest that the c.1378A>T ANGPTL6 variant leads to effective expression of the truncated Lys460Ter-ANGPTL6 protein, which is not secreted. Accordingly, heterozygous carriers for the c.1378A>T ANGPTL6 variant are expected to present with decreased levels of circulating ANGPTL6. To assess this hypothesis, we performed ELISA to compare the serum concentration of ANGPTL6 in subjects from family A reported as homozygous for the WT-ANGPTL6 (n=5) versus heterozygous for the c.1378A>T ANGPTL6 (n=7), and found a 50% reduction in the serum level of ANGPTL6 in heterozygous carriers (FIG. 2B).

Enrichment in Rare Coding Variants within ANGPTL6 Among IA Carriers

We then extended genetic screening on the coding portion of ANGPTL6 to 94 additional index cases with familial IA. We identified 5 additional individuals carrying rare, non-synonymous variants in ANGPTL6 predicted as damaging in silico by PolyPhen-2 and/or SIFT (data not shown): two cases with the same missense mutation in exon 1 leading to the p.Glu131Val substitution in ANGPTL6, one case with a missense mutation in exon 4 leading to the p.Leu348Phe substitution, and two cases carrying the same CGCGCTGAGCCTCGGCGGA-bp (SEQ ID NO: 1) insertion leading to one premature STOP codon in exon 2 (p.Ala153ValfsTer66). By ELISA, we found no reduction in the serum concentration of ANGPTL6 between p.Glu131Val heterozygous carriers versus non carriers (data not shown).

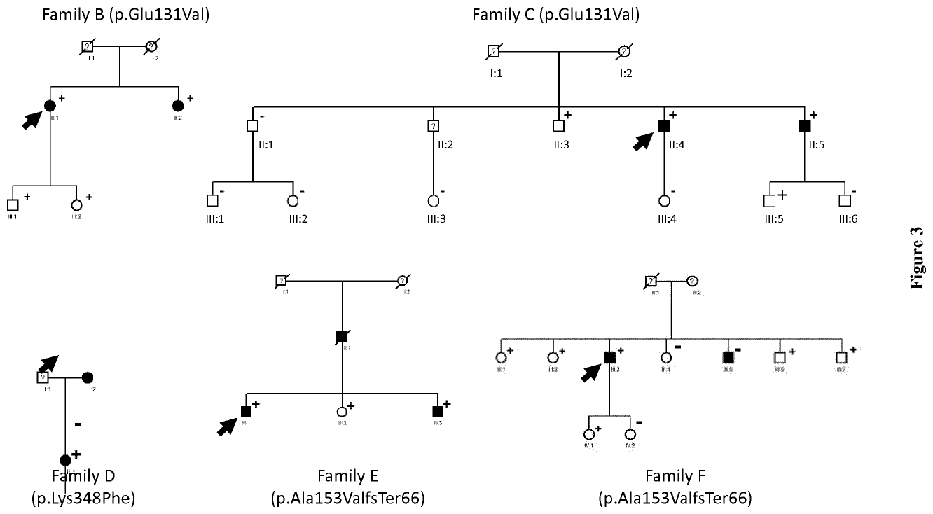

Overall, from the 6 index cases, family screening led to the identification of 16 relatives with diagnosed IA (FIG. 1 and FIG. 3). Out of the 13 family members carrying IA and agreeing to participate in genetic research, 12 (92%) carry rare coding variants in ANGPTL6, versus 15 out of 41 (34%) unaffected ones. The only affected individual who does not carry any rare coding variant in ANGPTL6 is a 54 year-old male (III-5, family F, FIG. 3) presenting with an aneurysm on the anterior communicant artery, with no reported history of smoking, high blood pressure or any relevant associated disease.

The clinical characteristics of the remaining 12 cases are studied. Seven of them (58%) carry multiple IA (with a maximum of three). IA is located on the middle cerebral artery bifurcation in 7 cases (58%), on the anterior communicant artery, the anterior cerebral artery and the internal carotid artery in 3 cases (25%), and on the posterior communicant artery in 2 cases (17%).

To further test the association of ANGPTL6 rare variants with susceptibility to familial IA, we also compared the proportions of individuals carrying at least one rare, non-synonymous variant across this gene among the 95 index cases enrolled in the present study (6/95; 6.32%) versus 404 healthy individuals with French ancestry (8/404; 1.98%). We found a significant enrichment in carriers of non-synonymous variants with an MAF below 1% in the NFE reference population, among IA cases (SKAT, p=0.023). Similar results were found when comparing allele counts among the 95 index cases versus the 7,509 Non-Finnish European individuals with whole-genome sequences available in the gnomAD database (data not shown).

### Example 2: Assessment of the Cerebral Vascular Phenotype of K447*Angplt6 Mice

Material & Methods

Angptl6 domains and sequence are highly conversed between humans and mice. K460 in the human Angptl6 sequence corresponds to K447 in the mouse sequence and the K447*Angplt6 mouse mutant also lacks the last 11 C-terminal residues. To assess the causal link between this Angptl6 variant and IA, inventors have generated a mouse model expressing the truncated form of Angptl6, analogue to the human mutation. The point mutation has been introduced into the Angptl6 gene sequence by homolog recombination. Mice express the K447*Angptl6 protein instead of the wild-type Angptl6 but the expression pattern and its regulation are not modified. Cerebral vasculature of heterozygous (Angpl6+/Δ) and homozygous (Angptl6Δ/Δ) mice have been analyzed and compared with control mice (Angpl6+/+).

Results:

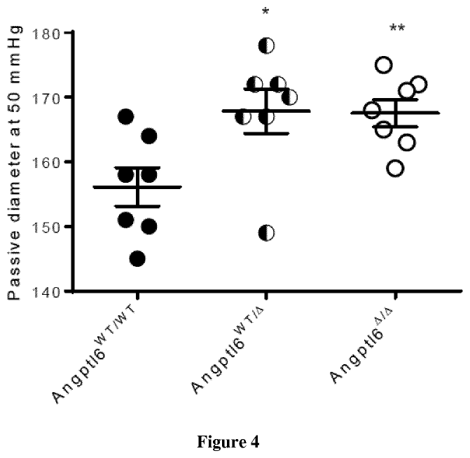

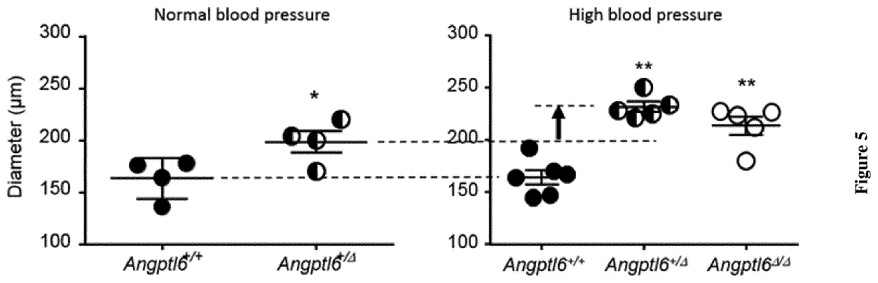

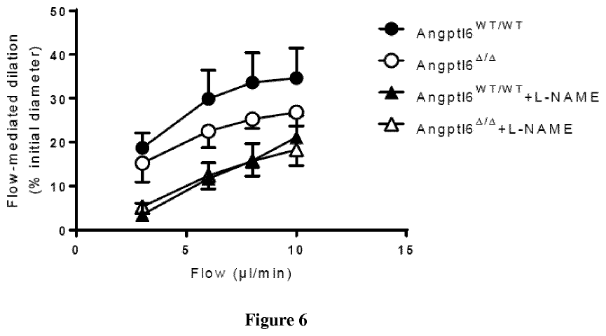

Inventors have observed: (i) an increased diameter of cerebral arteries of Angptl6 mutant mice compared to controls ex vivo (FIG. 4); (ii) with Micro-computed tomography (μCT) imaging of cerebral arteries in Angptl6+/+ and Angptl6 mutant mice showing increased arterial diameter in the mutant mice at normal systolic blood pressure (SBP: 104.7±2.9 mm Hg in Angptl6Δ/Δ mice and 103.1±4.2 mm Hg in Angpl6+/Δ; FIG. 5 (left)); (iii) a defect in the adaptation to high blood pressure leading to a dilation of cerebral arteries (FIG. 5 (right); SBP: 127.6±4.4 mm Hg in Angptl6Δ/Δ mice, 122.9±4.5 mm Hg in Angpl6+/Δ and 117.5±6.7 mm Hg in Angpl6 A/A), and (iv) reduced NO-dependent relaxation in response to flow (FIG. 6).

These results suggest that expression of the K447*Angptl6 variant leads to structural and functional defects of cerebral arteries, including endothelial dysfunction.

### REFERENCES

Throughout this application, various references describe the state of the art to which this invention pertains. The disclosures of these references are hereby incorporated by reference into the present disclosure.

1. Vlak M H, Algra A, Brandenburg R, Rinkel G J. Prevalence of unruptured intracranial aneurysms, with emphasis on sex, age, comorbidity, country, and time period: a systematic review and meta-analysis. Lancet Neurol. 2011 July; 10(7):626-36.

2. Laaksamo E, Ramachandran M, Frosen J, Tulamo R, Baumann M, Friedlander R M, et al. Intracellular signaling pathways and size, shape, and rupture history of human intracranial aneurysms. Neurosurgery. 2012 June; 70(6):1565-1572; discussion 1572-1573.

3. Nieuwkamp D J, Setz L E, Algra A, Linn F H H, de Rooij N K, Rinkel G J E. Changes in case fatality of aneurysmal subarachnoid haemorrhage over time, according to age, sex, and region: a meta-analysis. Lancet Neurol. 2009 July; 8(7):635-42.

4. Pierot L, Spelle L, Vitry F. Immediate clinical outcome of patients harboring unruptured intracranial aneurysms treated by endovascular approach: results of the ATENA study. Stroke. 2008 September; 39(9):2497-504.

5. Huttunen T, von and zu Fraunberg M, Frosen J, Lehecka M, Tromp G, Helin K, et al. Saccular intracranial aneurysm disease: distribution of site, size, and age suggests different etiologies for aneurysm formation and rupture in 316 familial and 1454 sporadic eastern Finnish patients. Neurosurgery. 2010 April; 66(4):631-638; discussion 638.

6. Bacigaluppi S, Piccinelli M, Antiga L, Veneziani A, Passerini T, Rampini P, et al. Factors affecting formation and rupture of intracranial saccular aneurysms. Neurosurg Rev. 2014 January; 37(1):1-14.

7. Frosen J. Smooth muscle cells and the formation, degeneration, and rupture of saccular intracranial aneurysm wall—a review of current pathophysiological knowledge. Transl Stroke Res. 2014 June; 5(3):347-56.

8. Vlak M H M, Rinkel G J E, Greebe P, Algra A. Independent risk factors for intracranial aneurysms and their joint effect: a case-control study. Stroke. 2013 April; 44(4):984-7.

9. Bourcier R, Redon R, Desal H. Genetic investigations on intracranial aneurysm: Update and perspectives. J Neuroradiol. 2015 Feb. 9;

10. Alg V S, Sofat R, Houlden H, Werring D J. Genetic risk factors for intracranial aneurysms: A meta-analysis in more than 116,000 individuals. Neurology. 2013 Jun. 4; 80(23):2154-65.

11. Yasuno K, Bakircio\check{g}lu M, Low S-K, Bilgüvar K, Gaáal E, Ruigrok Y M, et al. Common variant near the endothelin receptor type A (EDNRA) gene is associated with intracranial aneurysm risk. Proc Natl Acad Sci USA. 2011 Dec. 6; 108(49):19707-12.

12. Zhou S, Ambalavanan A, Rochefort D, Xie P, Bourassa C V, Hince P, et al. RNF213 Is Associated with Intracranial Aneurysms in the French-Canadian Population. The American Journal of Human Genetics. 2016 November; 99(5):1072-85.

13. Santiago-Sim T, Fang X, Hennessy M L, Nalbach S V, DePalma S R, Lee M S, et al. THSD1 (Thrombospondin Type 1 Domain Containing Protein 1) Mutation in the Pathogenesis of Intracranial Aneurysm and Subarachnoid Hemorrhage. Stroke. 2016 December; 47(12):3005-13.

14. Liu W, Morito D, Takashima S, Mineharu Y, Kobayashi H, Hitomi T, et al. Identification of RNF213 as a susceptibility gene for moyamoya disease and its possible role in vascular development. PLoS ONE. 2011; 6(7):e22542.

15. Kamada F, Aoki Y, Narisawa A, Abe Y, Komatsuzaki S, Kikuchi A, et al. A genome-wide association study identifies RNF213 as the first Moyamoya disease gene. J Hum Genet. 2011 January; 56(1):34-40.

16. Bourcier R, Chatel S, Bourcereau E, Jouan S, Marec H L, Daumas-Duport B, et al. Understanding the Pathophysiology of Intracranial Aneurysm: The ICAN Project. Neurosurgery. 2017 Apr. 1; 80(4):621-6.

17. Li H, Durbin R. Fast and accurate long-read alignment with Burrows-Wheeler transform. Bioinformatics. 2010 Mar. 1; 26(5):589-95.

18. McKenna A, Hanna M, Banks E, Sivachenko A, Cibulskis K, Kernytsky A, et al. The Genome Analysis Toolkit: a MapReduce framework for analyzing next-generation DNA sequencing data. Genome Res. 2010 September; 20(9):1297-303.

19. Lindenbaum P, Le Scouarnec S, Portero V, Redon R. Knime4Bio: a set of custom nodes for the interpretation of next-generation sequencing data with KNIME. Bioinformatics. 2011 Nov. 15; 27(22):3200-1.

20. Han L, Abney M. Identity by descent estimation with dense genome-wide genotype data. Genetic Epidemiology. 2011; n/a-n/a.

21. Karakachoff M, Duforet-Frebourg N, Simonet F, Le Scouarnec S, Pellen N, Lecointe S, et al. Fine-scale human genetic structure in Western France. Eur J Hum Genet. 2015 June; 23(6):831-6.

22. Wu M C, Lee S, Cai T, Li Y, Boehnke M, Lin X. Rare-variant association testing for sequencing data with the sequence kernel association test. Am J Hum Genet. 2011 Jul. 15; 89(1):82-93.

23. Morgenthaler S, Thilly W G. A strategy to discover genes that carry multi-allelic or mono-allelic risk for common diseases: a cohort allelic sums test (CAST). Mutat Res. 2007 Feb. 3; 615(1-2):28-56.

24. Lek M, Karczewski K J, Minikel E V, Samocha K E, Banks E, Fennell T, et al. Analysis of protein-coding genetic variation in 60,706 humans. Nature. 2016 18; 536(7616):285-91.

25. Thorvaldsdottir H, Robinson J T, Mesirov J P. Integrative Genomics Viewer (IGV): high-performance genomics data visualization and exploration. Briefings in Bioinformatics. 2013 Mar. 1; 14(2):178-92.

26. Hato T, Tabata M, Oike Y. The role of angiopoietin-like proteins in angiogenesis and metabolism. Trends Cardiovasc Med. 2008 January; 18(1):6-14.

27. Santulli G. Angiopoietin-like proteins: a comprehensive look. Front Endocrinol (Lausanne). 2014; 5:4.

28. Oike Y, Ito Y, Maekawa H, Morisada T, Kubota Y, Akao M, et al. Angiopoietin-related growth factor (AGF) promotes angiogenesis. Blood. 2004 May 15; 103(10):3760-5.

29. Romeo S, Yin W, Kozlitina J, Pennacchio L A, Boerwinkle E, Hobbs H H, et al. Rare loss-of-function mutations in ANGPTL family members contribute to plasma triglyceride levels in humans. J Clin Invest. 2009 January; 119(1):70-9.

30. Leblanc G G, Golanov E, Awad I A, Young W L, Biology of Vascular Malformations of the Brain NINDS Workshop Collaborators. Biology of vascular malformations of the brain. Stroke. 2009 December; 40(12):e694-702.

31. Hellström M, Kalen M, Lindahl P, Abramsson A, Betsholtz C. Role of PDGF-B and PDGFR-beta in recruitment of vascular smooth muscle cells and pericytes during embryonic blood vessel formation in the mouse. Development. 1999 June; 126(14):3047-55.

32. Raab S., Beck H., Gaumann A., Yuce A., Gerber H. P., Plate K., Hammes H. P., Ferrara N., Breier G. Impaired brain angiogenesis and neuronal apoptosis induced by conditional homozygous inactivation of vascular endothelial growth factor. Thromb. Haemost. 2004; 91:595-605.

33. Nguyen H L, Lee Y J, Shin J, Lee E, Park S O, McCarty J H, Oh S P. TGF-β signaling in endothelial cells, but not neuroepithelial cells, is essential for cerebral vascular development. Lab Invest. 2011 November; 91(11):1554-63.

34. Bourcier R, Lenoble C, Guyomarch-Delasalle B, Daumas-Duport B, Papagiannaki C, Redon R, et al. Is there an inherited anatomical conformation favoring aneurysmal formation of the anterior communicating artery? J Neurosurg. 2017 May; 126(5): 1598-605.

35. Mackey J, Brown R D Jr, Moomaw C J, Hornung R, Sauerbeck L, Woo D, et al. Familial Intracranial Aneurysms: Is Anatomic Vulnerability Heritable? Stroke. 2013 January; 44(1):38-42.

36. Oike Y, Akao M, Yasunaga K, Yamauchi T, Morisada T, Ito Y, et al. Angiopoietin-related growth factor antagonizes obesity and insulin resistance. Nat Med. 2005 April; 11(4):400-8.

1. A method for predicting the risk of having or developing Intracranial aneurysms (IA) in a subject, comprising the steps of: i) identifying at least one mutation in an angiogenic protein; and ii) concluding that the subject is at risk of having or developing IA when at least one mutation is identified in said angiogenic protein.

2. The method according to claim 1, wherein, the angiogenic protein is angiopoietin-Like 6 (ANGPTL6).

3. A method for predicting the risk of having or developing Intracranial aneurysms (IA) in a subject comprises following steps: i) determining the expression level of ANGPTL6 in a biological sample obtained from said subject, ii) comparing the expression level of ANGPTL6 determined at step i) with a predetermined reference value and iii) concluding that the subject is at risk of having or developing IA when the expression level of ANGPTL6 determined at step i) is lower than the predetermined reference value, or concluding that the patient is not at risk of having or developing IA when the expression level of ANGPTL6 determined at step i) is higher than the predetermined reference value.

4. The method according to claim 1, wherein the method further comprises a step of detecting an angiogenic single nucleotide polymorphism (SNP) in a biological sample obtained from said subject.

5. A method of treating and/or preventing intracranial aneurysms in a subject in need thereof, comprising, i) determining the expression level of ANGPTL6 an angiogenic protein in a biological sample obtained from said subject, ii) comparing the expression level of ANGPTL6 the angiogenic protein determined at step i) with a predetermined reference value and iii) treating the subject by surgery and/or by one or more endovascular techniques when the expression level of the an angiogenic protein determined at step i) is lower than the predetermined reference value.

6. A kit for performing the methods according to claim 1, wherein said kit comprises means for measuring the expression level of an angiogenic protein and/or detecting angiogenic SNP that is indicative of subject at risk of having or developing Intracranial aneurysms (IA).

7. The method according to claim 5, wherein the angiogenic protein is angiopoietin-like 6 (ANGPTL6).

8. The method according to claim 5, further comprising a step of detecting a single nucleotide polymorphism (SNP) in a gene encoding the angiogenic protein.


SEQUENCE LISTING (1 sequence)

SEQ ID NO: 1 - DNA, 19 bp, Homo sapiens
cgcgctgagcctcggcgga